(19) United States
(12) Patent Application Publication
(10) Pub. No.: US 2011/0305680 A1
(43) Pub. Date: Dec. 15, 2011

(54) Composition Containing Osteopontin for Differentiating Natural Killer Cell as an Active Ingredient and a Method of Differentiation Using Thereof

(75) Inventors: Inpyo Choi, Daejeon (KR); Jin Woong Chung, Daejeon (KR); Misun Kim, Daejeon (KR); Suk Ran Yoon, Daejeon (KR)

(73) Assignee: KOREA RESEARCH INSTITUTE OF BIOSCIENCE AND BIOTECHNOLOGY, Taejeon-si (KR)

(21) Appl. No.: 12/995,020

(22) PCT Filed: Nov. 10, 2008
(86) PCT No.: PCT/KR2008/006612

§ 371 Date: Nov. 29, 2010

(30) Foreign Application Priority Data
May 27, 2008 (KR) 10-2008-0049034

(51) Int. Cl. A61K 35/12; A61P 35/00; C12N 5/0783

(52) U.S. Cl. 424/93.71; 435/377

(57) ABSTRACT

The present invention relates to a composition for differentiating natural killer cells comprising osteopontin (OPN) as an active ingredient and a method for differentiation using the same. More precisely, osteopontin of the present invention accelerates differentiation of natural killer cells from hematopoietic stem cells and increases cytotoxic activity of natural killer cells, so that it can be effectively used as a composition for differentiating natural killer cells. OPN of the present invention regulates differentiation of natural killer cells capable of killing cancer cells, so that it can be effectively used for the treatment of cancer.

### TECHNICAL FIELD

The present invention relates to a composition for accelerating differentiation of natural killer cells from stem cells and a method of differentiation using the same, more precisely a composition comprising osteopontin (OPN) as an active ingredient for differentiation of natural killer cells and a method for differentiation of natural killer cells from hematopoietic stem cells using the same.

### BACKGROUND ART

Natural killer cells (referred as “NK cells” hereinafter) are immune cells differentiated from hematopoietic stem cells in bone marrow. NK cells are able to kill those cells infected by a foreign antigen directly or indirectly by hiring other immune cells by secreting cytokines or chemokines. So, NK cells play a very important role in mediating acquired immune response of natural immune response.

While being differentiated, NK cells express a specific receptor on the cell surface. So, NK cell differentiation can be confirmed by investigating the receptor. Premature NK cells contain CD122, well known as IL2/1L15 receptor β. In the early stage of NK cell differentiation, important signals of IL15 cytokine are delivered by the CD122 receptor. By those signals, NK cells are differentiated, during which NK1.1, DX5 and Ly49 having diverse functions are expressed. Bone marrow contains the cells secreting many growth factors and cytokines necessary for the NK cell differentiation. As an example, stromal cells are involved in maturation of NK cells.

NK cells have non-specific cancer cell killing activity. Any defect in NK cell differentiation and activation causes diverse diseases including breast cancer, melanoma and lung cancer. To treat these diseases, NK cell based therapy has been proposed.

Osteopontin (OPN) is a glycoprotein, which is largely generated from stromal cells, the osteocytes, and then phosphorylated in many ways.

Osteopontin is expressed from activated T cells or plasmacytoid DC, which is regulated by the transcription factor T-bet. T-bet is conjugated to CD122 promoter to regulate CD122 expression. This mechanism is related to IL15 signal transduction and also affects CD8+ memory cells and NK cells expressing CD122.

Osteopontin has been known as a noncollagenous bone matrix protein in its early days, but is recognized as the protein involved importantly in immune system, particularly in regulation of cytokine secretion or cell migration (Ashkar, S. et al., Science, 287:860-864, 2000; Iizuka, J., et al., Laboratory investigation 78:1523-1533, 1998; Weber, G. F., et al., Cytokine & growth factor reviews, 7:241-248, 1996). In particular, osteopontin is known as a key factor involved in differentiation of CD4 T cells into TH1 during immune response (A. C. Renkl, et al., Blood, 106:946-955, 2005; Li, X., et al., J Interferon Cytokine Res, 23:259-265, 2003). However, there are no reports saying that osteopontin affects differentiation of natural killer cells.

The present inventors confirmed that osteopontin accelerated differentiation of natural killer cells from hematopoietic stem cells and could increase killing ability of natural killer cells, and thus the inventors completed this invention by further confirming that osteopontin could be used as a composition for differentiation of natural killer cells.

### DISCLOSURE

### Technical Problem

It is an object of the present invention to provide a composition comprising osteopontin (OPN) as an active ingredient for differentiation of natural killer cells and a method for differentiation of NK cells using the same.

### Technical Solution

To achieve the above object, the present invention provides a composition comprising osteopontin (OPN) as an active ingredient for differentiation of natural killer cells.

The present invention also provides a composition comprising osteopontin as an active ingredient for prevention and treatment of cancer.

The present invention further provides a method for accelerating differentiation of NK cells from hematopoietic stem cells containing the step of administering osteopontin to premature NK cells.

The present invention also provides a method for differentiation of NK cells having improved cytotoxic activity containing the step of administering osteopontin to premature NK cells.

The present invention also provides a method for treating cancer containing the step of administering NK cells differentiated by the said method and having improved cytotoxic activity to a subject with cancer.

The present invention also provides a method for preventing cancer containing the step of administering NK cells differentiated by the said method and having improved cytotoxic activity to a subject.

The present invention also provides an immune enhancing functional health food containing osteopontin as an active ingredient.

In addition, the present invention provides a functional health food for prevention of cancer and improvement of health conditions containing osteopontin as an active ingredient.

### Advantageous Effect

Osteopontin (OPN) of the present invention accelerates NK cell differentiation and increases cytotoxic activity thereof. Therefore the said osteopontin can be effectively used as a composition for differentiation of NK cells. Osteopontin of the present invention can also be used for cancer treatment by regulating differentiation of NK cells having cancer cell killing activity.

### DESCRIPTION OF DRAWINGS

The application of the preferred embodiments of the present invention is best understood with reference to the accompanying drawings, wherein:

FIG. 1 is a diagram illustrating the maturation of NK cells from hematopoietic stem cells (HSC) separated from mouse bone marrow cells:

pNK: premature NK cells; and

mNK: mature NK cells.

FIG. 2 is a diagram illustrating the expressions of differentiated NK cells of both the experimental group treated with the recombinant protein osteopontin (OPN) and the control group not-treated with OPN, investigated by flow cytometric analysis.

FIG. 3 is a diagram illustrating the inhibitory effect of an antibody on OPN that differentiates NK cells.

FIG. 4 is a diagram illustrating the expressions of differentiated NK cells in the experimental group treated with OPN and in the control group not-treated with OPN, analyzed by PCR and real-time PCR.

FIG. 5 is a diagram illustrating the activation of NK cells differentiated by OPN, analyzed by ⁵¹Cr release assay.

FIG. 6 is a diagram illustrating NK cells in OPN knock-out mouse bone marrow and spleen cells, analyzed by FACS.

FIG. 7 is a diagram illustrating the comparison of expression of LY49s largely expressed in mature NK cells between a wild-type mouse and an OPN knock-out mouse.

FIG. 8 is a diagram illustrating the result of real-time PCR analyzing the molecules expressed specifically in NK cells.

FIG. 9 is a diagram illustrating the result of real-time PCR investigating the OPN expression in the cells of each differentiation stage during NK cell differentiation in a mouse isolated by using FACSAria.

FIG. 10 is a diagram illustrating the result of ELISA investigating the OPN expression in the cells of each differentiation stage during NK cell differentiation in vitro from HSC isolated from mouse bone marrow.

FIG. 11A is a diagram illustrating the result of FACS, in which in vitro NK cell differentiation from OPN knock out mouse HSC is compared with that of the control.

FIG. 11B is a diagram illustrating the effect of OPN. HSC obtained from mouse bone marrow was cultured with wild-type mouse stromal cells and OPN knock-out mouse stromal cells. Then the effect of OPN from the stromal cells was examined.

FIG. 11C is a diagram illustrating the NK cell differentiation. HSC (donor cell) obtained from a wild-type mouse was transplanted into a wild-type mouse and an OPN knock-out mouse (recipient). After 6 weeks of differentiation, NK cell differentiation was investigated.

FIG. 11D is a diagram illustrating the NK cell differentiation. HSC (donor cell) obtained from a wild-type mouse or an OPN knock-out mouse was transplanted into a wild-type mouse (recipient) to induce NK cell differentiation.

FIG. 12 is a diagram illustrating the result of RT-PCR investigating the T-bet expression during in vitro NK cell differentiation from HSC obtained from mouse bone marrow.

FIG. 13 is a diagram illustrating the results of RT-PCR and real-time PCR investigating the T-bet expression in OPN knock-out mouse bone marrow.

FIG. 14 is a diagram illustrating the result of promoter assay investigating the cd122 expression induced by T-bet.

FIG. 15 is a diagram illustrating the result of FACS. NK cell differentiation from HSC obtained from a T-bet knock-out mouse was compared with that of the control and the recombinant OPN was treated thereto to induce NK differentiation, during which T-bet involvement was investigated.

FIG. 16 is a diagram illustrating the result of FACS. The cells under the differentiation were directly obtained from T-bet knock-out mouse bone marrow and the CD122 expression in the cells was compared with that of the control, to which the recombinant OPN was treated to induce NK differentiation, during which T-bet involvement was investigated.

FIG. 17 is a diagram illustrating the result of FACS. NK cells were separated from spleen of each wild-type mouse, T-bet knock-out mouse and OPN knock-out mouse. The cells which were not completely differentiated yet but under the differentiation process were obtained and the CD122 expressions therein were analyzed by FACS.

### BEST MODE

Hereinafter, the present invention is described in detail.

The present invention provides a composition for differentiating natural killer cells containing osteopontin (OPN) as an active ingredient.

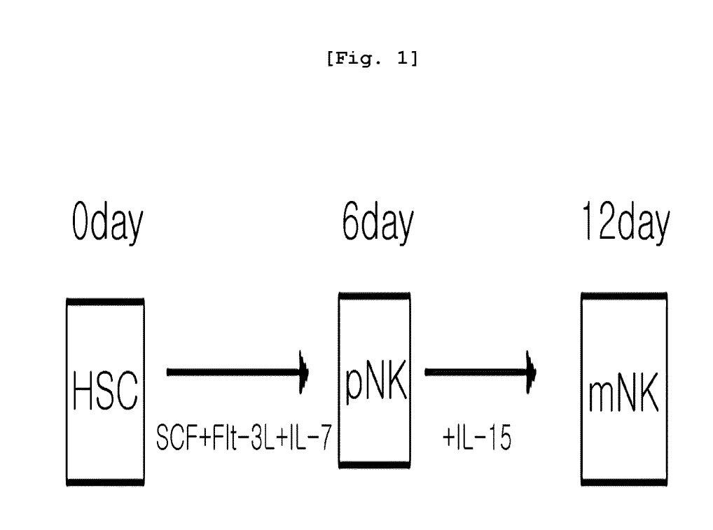

The present inventors obtained bone marrow cells from bones of lower limb of a mouse, from which CD117+ hematopoietic stem cells (referred as “HSC cells” hereinafter) were isolated by MACS (magnetic activated cell sorting). The isolated HSCs were differentiated to mature NK cells (mNK) through premature natural killer cells (pNK) (see FIG. 1).

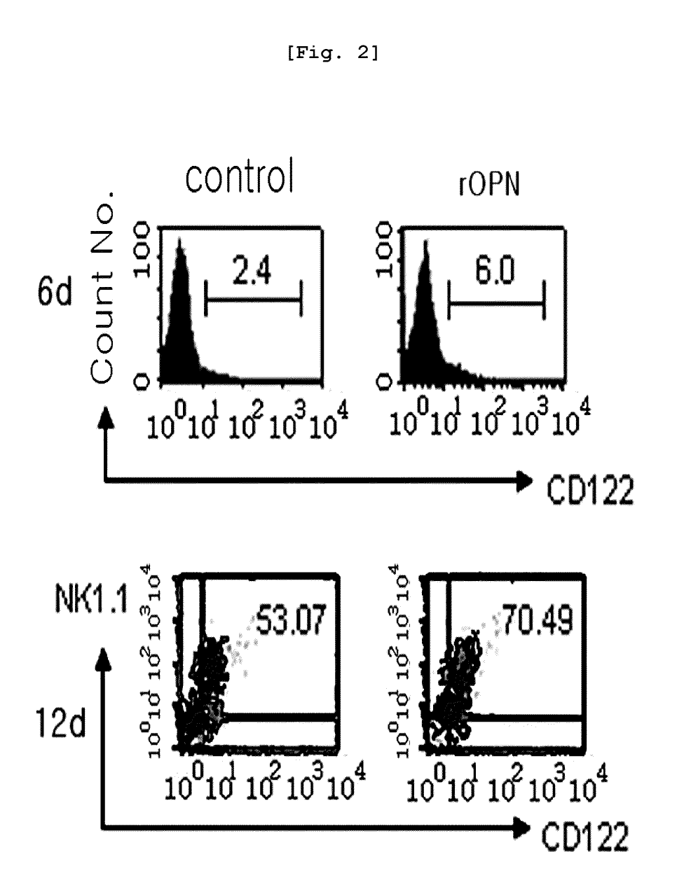

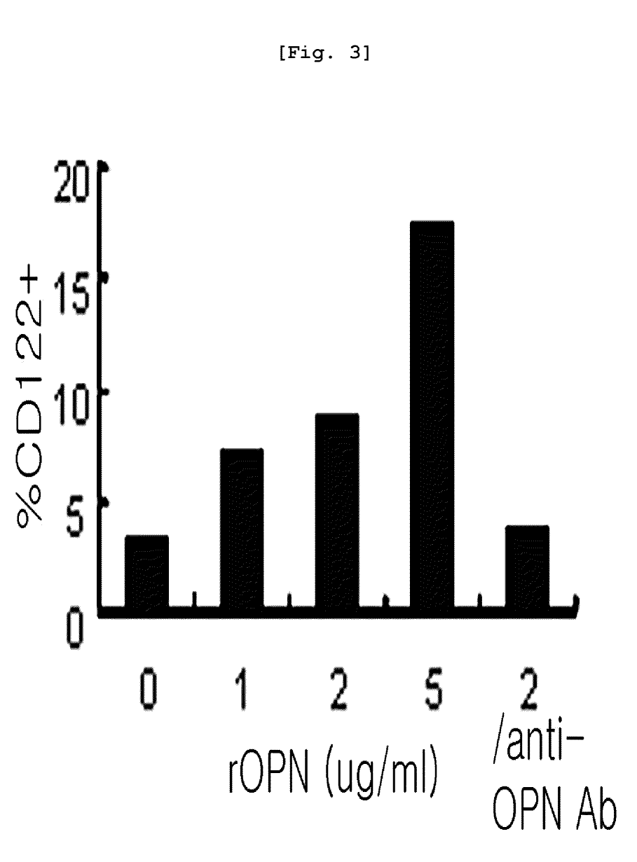

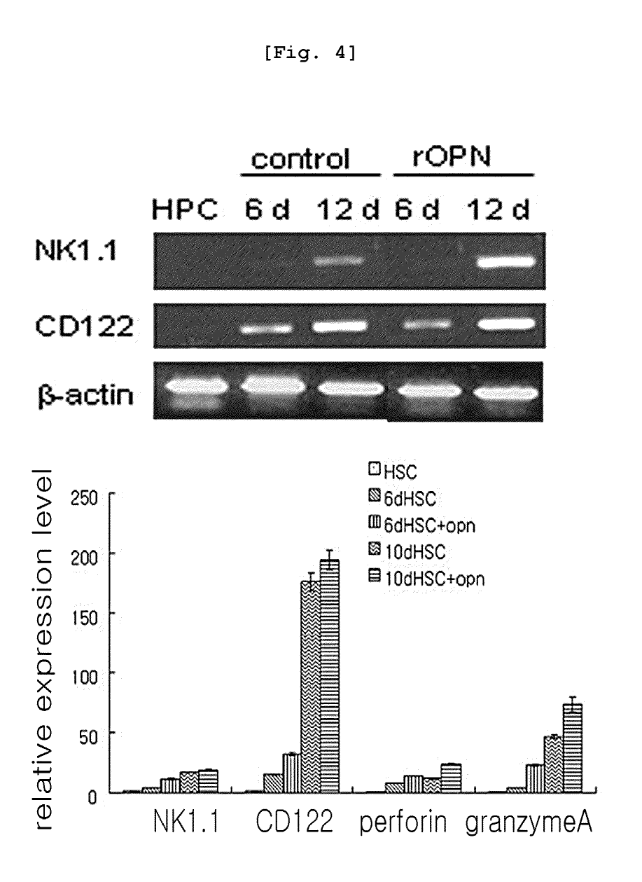

To investigate the effect of OPN on NK differentiation, the present inventors treated OPN to pNK during in vitro differentiation of NK from HSC, and then investigated differentiated mNK by FACS. As a result, the expression of NK was increased in the group treated with OPN, compared with the non-treated control (see FIG. 2). To investigate whether the above result indicated the recombinant OPN specific reaction, the recombinant OPN was inhibited by using an anti-OPN antibody (see FIG. 3). In addition, the expression patterns of NK related molecules such as CD122 and NK1.1 affected by recombinant OPN were investigated at RNA level by real-time PCR. As a result, CD122 and NK1.1 were up-regulated by recombinant OPN (see FIG. 4). Therefore, the above result indicates that OPN is involved in NK differentiation.

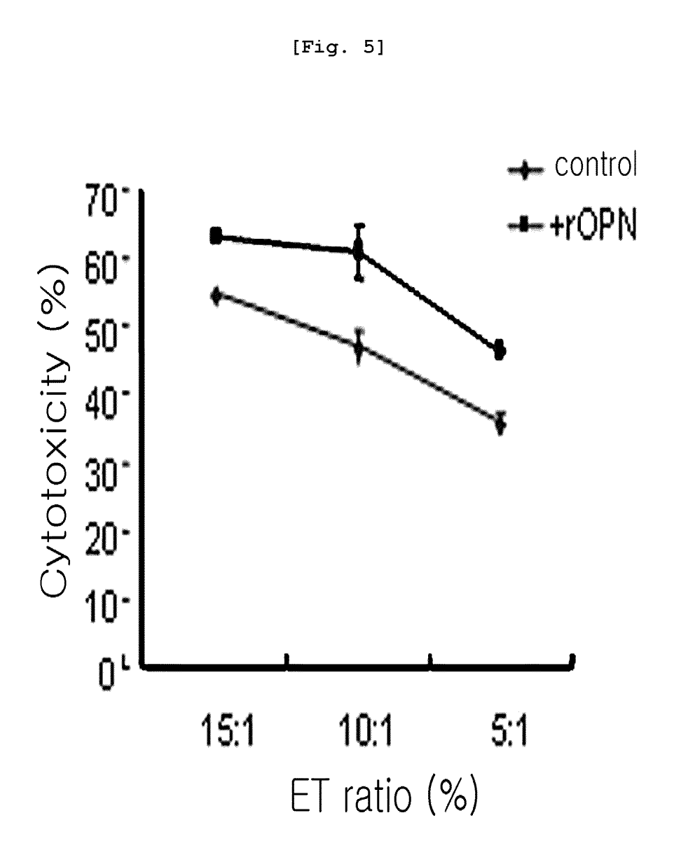

To investigate the effect of OPN on cytotoxic activity of NK, NK cells cultured above was co-cultured with ⁵¹Cr-labeled YAC-1 cells, the target cells, followed by examination of cytotoxicity by using γ-counter. As a result, cytotoxic activity of NK cells was increased by recombinant OPN (see FIG. 5). The above result indicates that OPN is involved in NK activation.

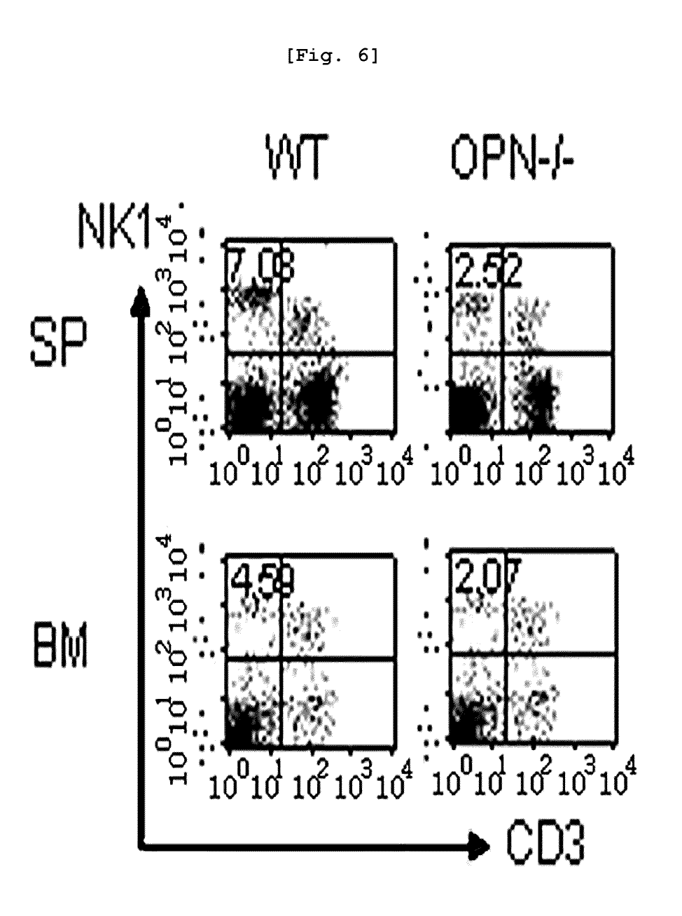

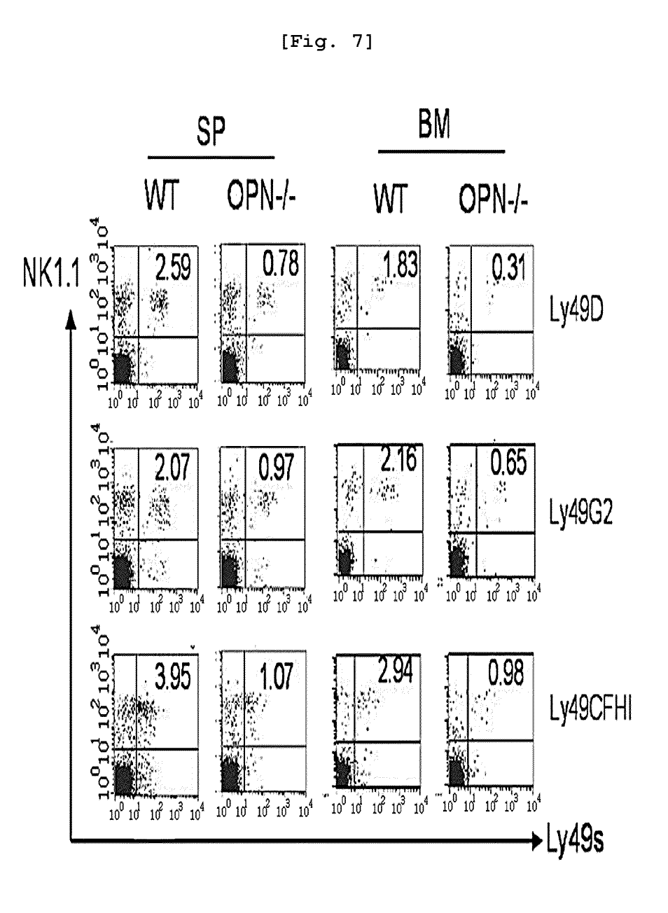

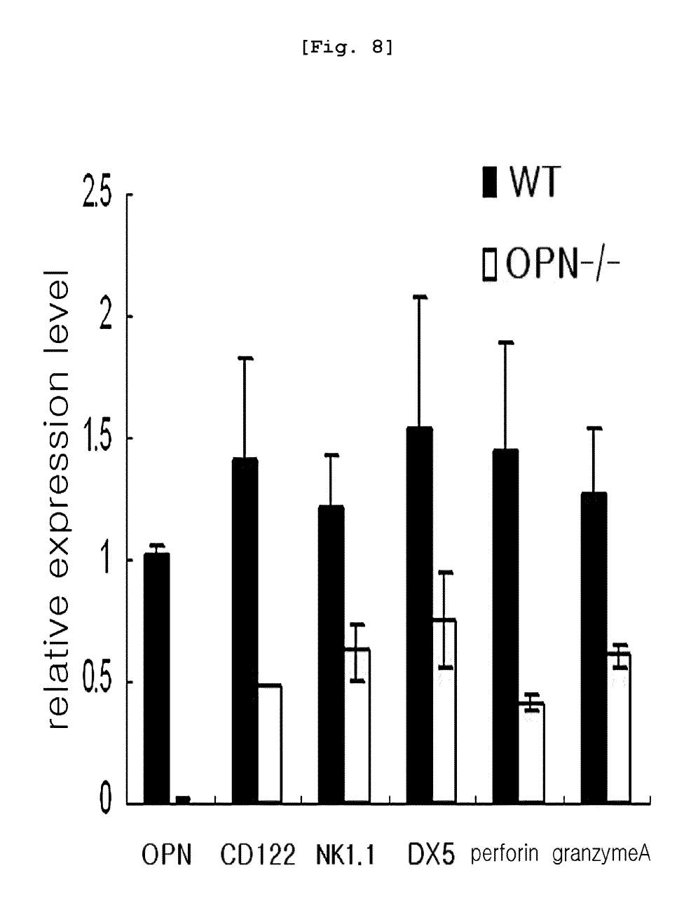

To investigate the effect of OPN deficiency on NK cells, the present inventors performed FACS with OPN knock-out mouse. As a result, NK cells were decreased in spleen and bone marrow of OPN knock-out mouse (see FIG. 6). The expression of LY49 receptor, the molecule largely expressed in mature NK cells, was significantly reduced in OPN-knock-out mouse (see FIG. 7). In addition, real-time PCR was performed to investigate expressions of NK cell specific molecules at RNA level. As a result, expressions of perforin and granzyme, both related to NK cells, were decreased (see FIG. 8). The above results indicate that OPN deficiency results in NK inhibition.

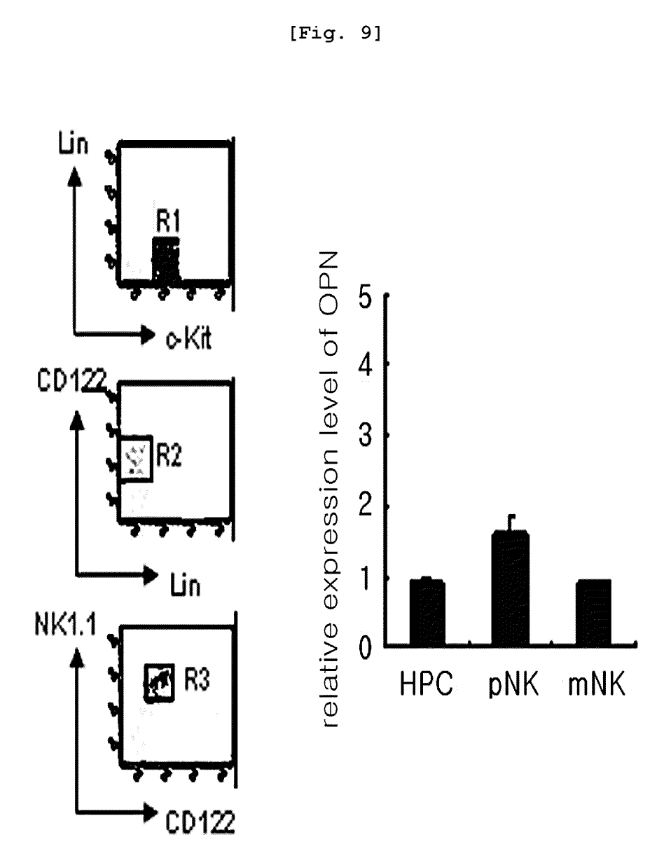

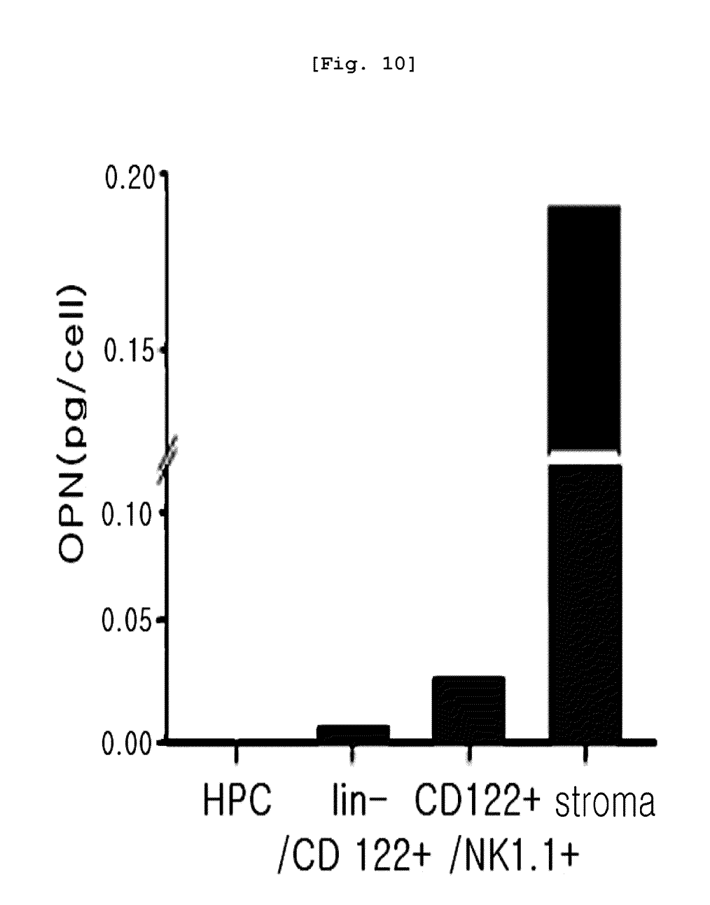

To investigate OPN expression in the cells under different differentiation stages in vitro and in vivo, real-time PCR and ELISA were performed. As a result, OPN expression was not much different by each differentiation stage in vivo (FIG. 9), while OPN was increased in mNK in vitro. And OPN expression was significantly increased in stromal cells (see FIG. 10). The above results indicate that intracellular OPN expression does not affect NK differentiation but OPN expression in stromal cells that is one of relevant environments affecting NK differentiation was high enough to affect NK cell differentiation.

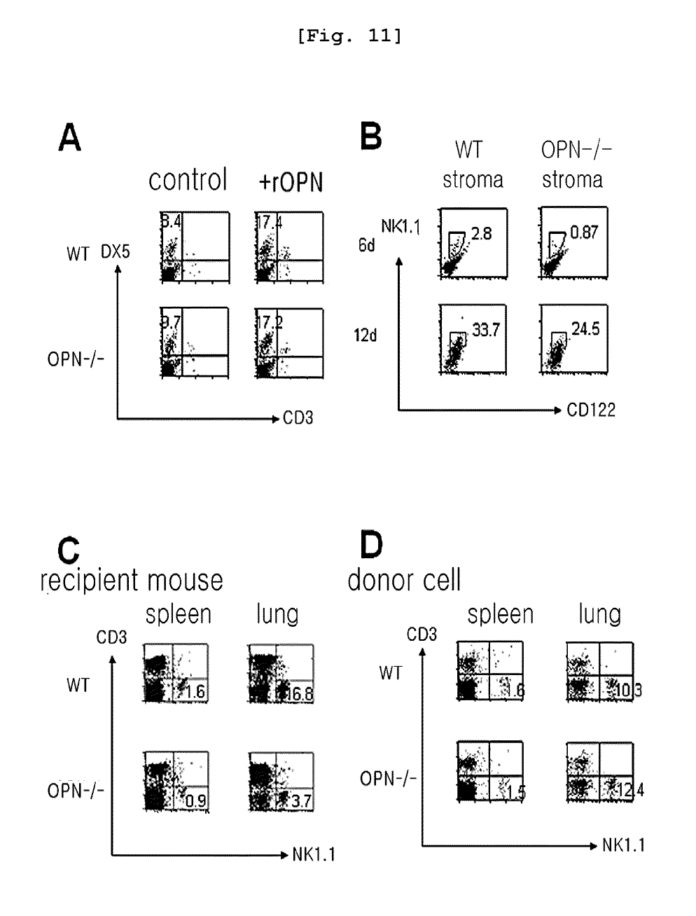

To investigate the origin of OPN that affects NK differentiation, the present inventors performed FACS to confirm NK differentiation. Differentiation of HSC separated from OPN knock-out mouse was increased by recombinant OPN (see FIG. 11A), while NK differentiation from HSC co-cultured with OPN knock-out stromal cells was inhibited (see FIG. 11B). Differentiation from HSC to NK in OPN knock-out mouse was significantly reduced in the mouse spleen and lung (see FIG. 11C). However, when HSC separated from OPN knock-out mouse and the control HSC were administered to a wild-type mouse, differentiation was not much changed (see FIG. 11D). So, it was confirmed that NK cell differentiation was affected by extracellular OPN.

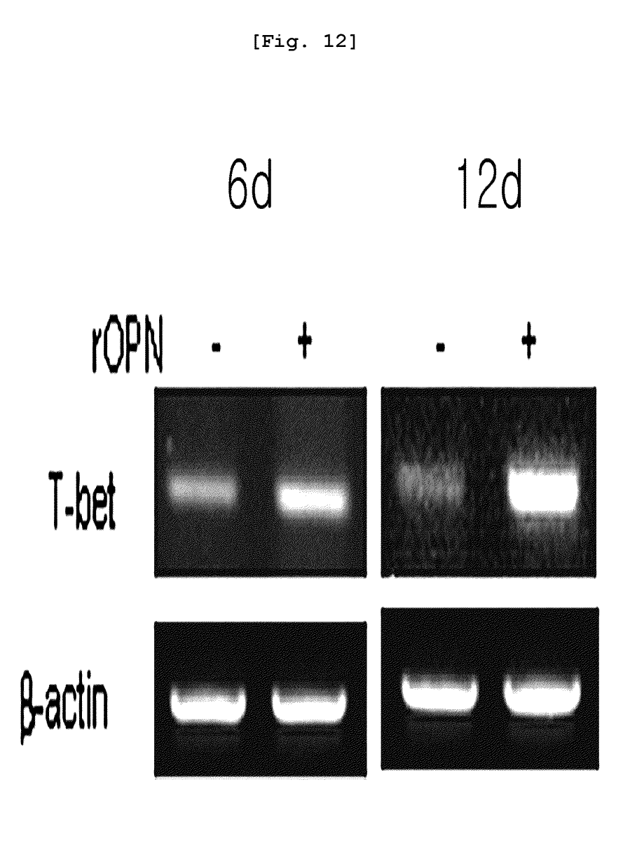

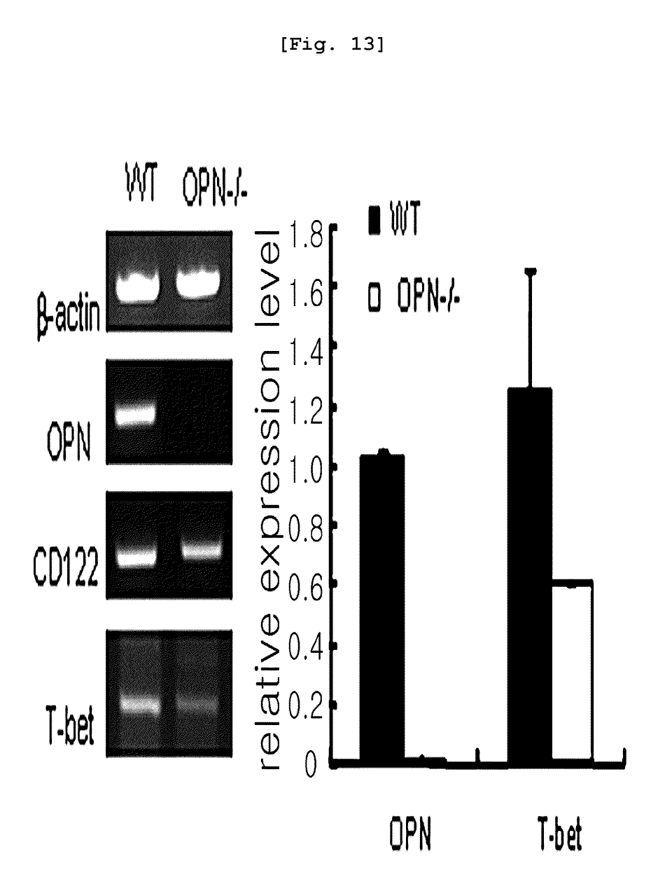

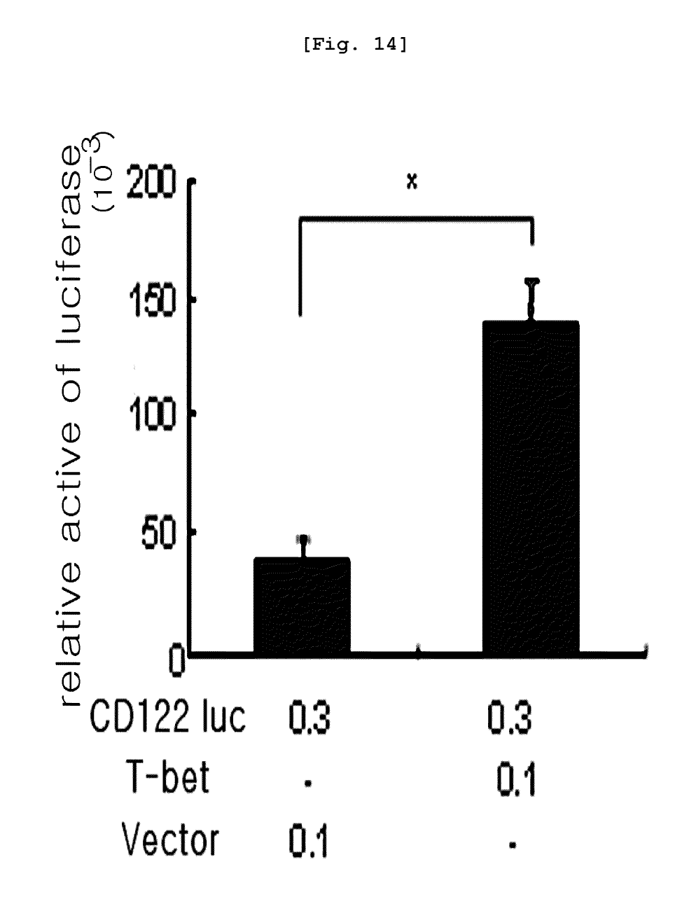

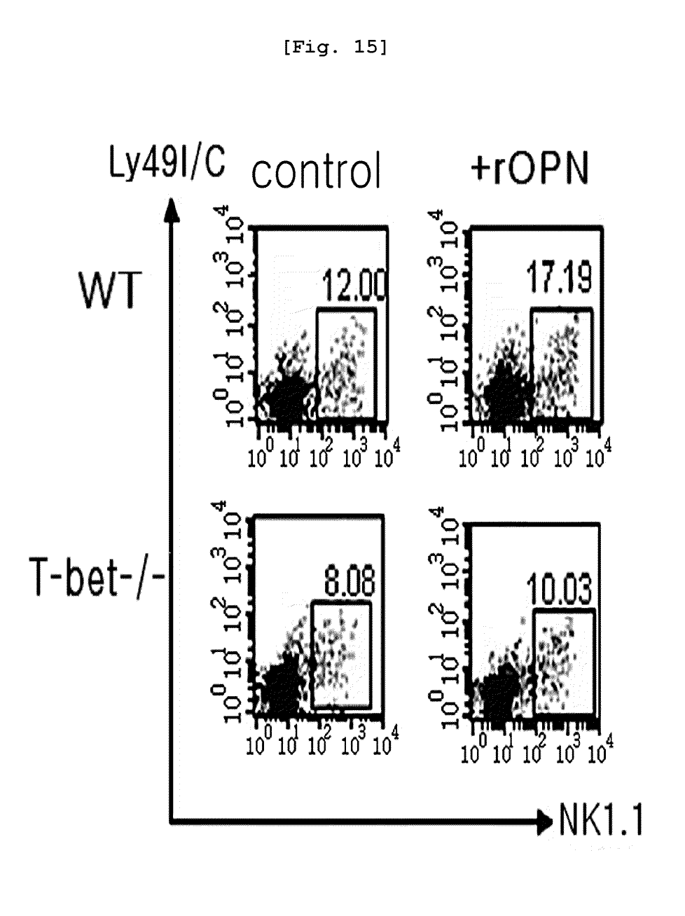

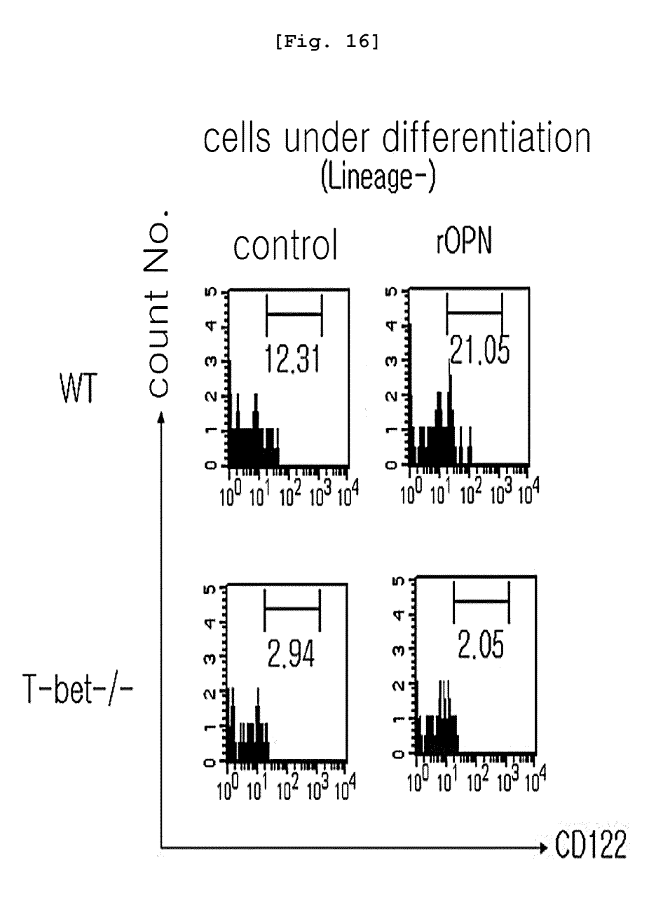

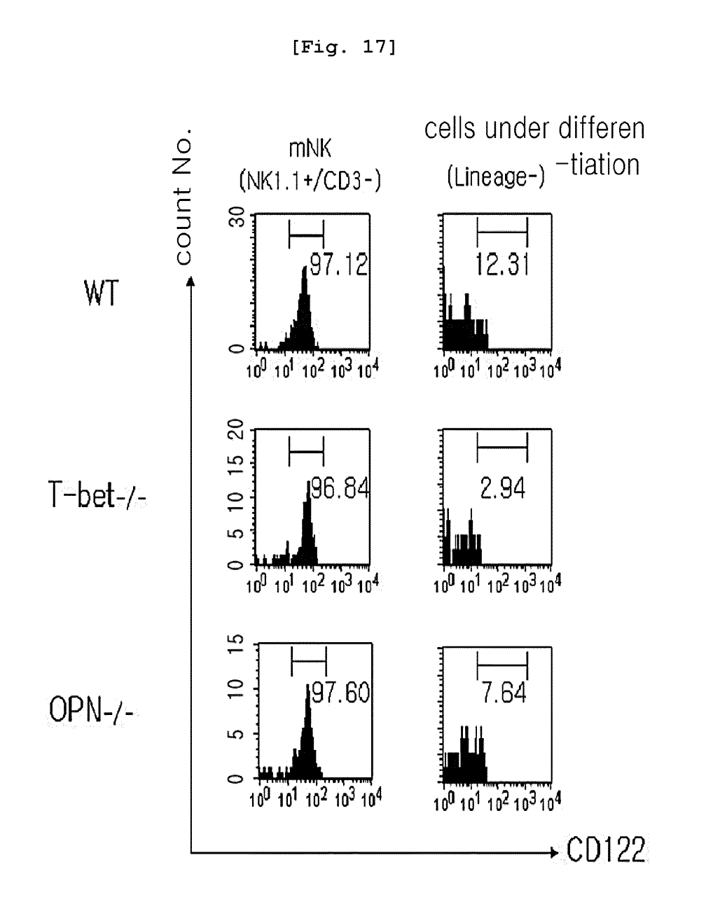

To search molecules necessary for OPN to affect NK differentiation, real-time PCR and RT-PCR were performed to investigate expression patterns of OPN and T-bet during NK differentiation. As a result, it was confirmed at RNA level that expression of T-bet was increased by the treatment of recombinant OPN (see FIG. 12). In the meantime, expression of T-bet was reduced in OPN knock-out mouse (see FIG. 13). CD122 expression was regulated by T-bet treatment, confirmed by promoter assay (see FIG. 14). In conclusion, T-bet increased by OPN could regulate CD122 expression to reinforce NK cell differentiation. To confirm the said conclusion, HSC separated from T-bet knock-out mouse was differentiated. As a result, OPN effect was not detected (see FIG. 15). Also, OPN effect was not observed in non-differentiated cells separated from a wild-type mouse (see FIG. 16). Therefore, it was confirmed that OPN affects NK differentiation via T-bet and OPN does not affect mature NK cells that had already been through differentiation but affects those cells not differentiated yet (see FIG. 17).

As explained hereinbefore, OPN accelerates NK cell differentiation from HSC and increases cytotoxic activity of NK cells, so that OPN can be effectively used as a composition for differentiating NK cells.

The present invention also provides a composition comprising osteopontin as an active ingredient for preventing and treating cancer.

The cancer herein is not limited but preferably selected from the group consisting of breast cancer, melanoma, stomach cancer and lung cancer.

Once there is a defect in NK cell differentiation and activation, diverse cancers are developed, for example, breast cancer (Konjevic G, et al., Breast Cancer Res. Treat., 66: 255-263, 2001), melanoma (Ryuke Y, et al., Melanoma Res., 2003, 13: 349-356) and lung cancer (Villegas F R, et al., Lung Cancer, 35: 23-28, 2002) can be developed. OPN promotes NK cell differentiation and increases cytotoxic activity thereof, so that it can be applied in a composition for preventing and treating cancer.

The composition of the present invention can include, in addition to osteopontin, one or more effective ingredients having the same or similar function to osteopontin. The composition of the present invention can include one or more pharmaceutically acceptable carriers such as saline, sterilized water, Ringer's solution, buffered saline, dextrose solution, maltodextrin solution, glycerol, ethanol, and a mixture comprising one or more of those components. If necessary, a general additive such as an antioxidant, a buffer and a bacteriostatic agent can be additionally added. The composition of the present invention can be formulated in different forms including aqueous solutions, suspensions and emulsions for injection, pills, capsules, granules or tablets by mixing with diluents, dispersing agents, surfactants, binders and lubricants. The composition can further be prepared in suitable forms according to ingredients by following the method represented in Remington's Pharmaceutical Science (Mack Publishing Company, Easton Pa., 18th, 1990).

The composition of the present invention can be administered orally or parenterally (for example, intravenous, hypodermic, peritoneal or local injection). The effective dosage of the composition can be determined according to weight, age, gender, health condition, diet, administration frequency, administration method, excretion and severity of a disease. The dosage is 0.015000 mg/kg per day and preferably 0.0110 mg/kg per day, and administration frequency is once a day or preferably a few times a day.

The present invention further provides a method for accelerating differentiation of NK cells from hematopoietic stem cells comprising the following steps:

1) inducing proliferation of pNK by adding a pNK inducer to HSC; and

2) inducing differentiation to mature NK cells by adding OPN to the pNK of step 1).

In this method, the pNK inducer of step 1) is a material that is able to induce differentiation of pNK from HSC, which is preferably SCF of Flt3L, but not always limited thereto.

In this method, the pNK of step 2) is preferably co-cultured with IL-15 (Interleukin-15), but not always limited thereto.

As explained hereinbefore, OPN accelerates differentiation of NK from HSC and increases cytotoxic activity of NK. So, the above method facilitates promotion of NK cell differentiation from HSC and enhancement of cytotoxic activity of differentiated NK cells.

The present invention also provides a method of differentiating NK cells with improved cytotoxic effect comprising the following steps:

1) inducing proliferation of pNK by adding a pNK inducer to HSC; and

2) inducing differentiation to mature NK cells by adding OPN to the pNK of step 1).

The present invention also provides a method for treating cancer containing the step of administering NK cells differentiated by the said method and having improved cytotoxic activity to a subject with cancer.

The present invention also provides a method for preventing cancer containing the step of administering NK cells differentiated by the said method and having improved cytotoxic activity to a subject.

The cancer herein is not limited but preferably selected from the group consisting of breast cancer, melanoma, stomach cancer and lung cancer.

The present invention also provides immune enhancing functional health food containing OPN as an active ingredient.

The OPN herein enhances immunity by accelerating differentiation of NK and increasing cytotoxic activity of NK, but not always limited thereto.

In addition, the present invention provides functional health food for prevention of cancer and improvement of health conditions containing OPN as an active ingredient.

The OPN of the present invention can be used as a food additive. In that case, OPN can be added as it is or as mixed with other food components according to the conventional method.

The mixing ratio of active ingredients can be regulated according to the purpose of use (prevention, health enhancement or treatment). In general, to produce health food or beverages, the OPN of the present invention is added preferably by 0.01˜10 weight part and more preferably by 0.05˜1 weight part. However, if long term administration is required for health and hygiene or regulating health condition, the content can be lower than the above but higher content can be accepted as well since the OPN of the present invention has been proved to be very safe.

The food herein is not limited. For example, the OPN of the present invention can be added to meat, sausages, bread, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

The composition for health beverages of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents such as thaumatin and stevia extract, and synthetic sweetening agents such as saccharin and aspartame can be included as a sweetening agent. The content of the natural carbohydrate is preferably 0.01˜0.04 g and more preferably 0.02˜0.03 g in 100 ml of the composition.

In addition to the ingredients mentioned above, the OPN of the present invention can include in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The OPN of the present invention can also include natural fruit juice, fruit beverages and/or fruit flesh addable to vegetable beverages. All the mentioned ingredients can be added independently or together. The mixing ratio of those ingredients does not matter in fact, but in general, each can be added by 0.01˜0.1 weight part per 100 weight part of the OPN of the present invention.

### Mode for Invention

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Separation of Hematopoietic Stem Cells from Bone Marrow Cells

Bones of lower limbs were obtained from 6-9 week old C57BL/6 mice (Coretech, Korea), which were grinned using a mortar. The pulverized bone cells were passed through 70 μm cell strainer, and then treated with lysis buffer (Sigma, St. Louse, Mo.) to eliminate erythrocytes. As a result, bone marrow cells were obtained. Already differentiated cells were eliminated from the obtained bone marrow cells and rest of the cells were obtained by negative selection using MACS (magnetic activated cell sorting) (Miltenyi Biotech, Auburn, Calif.). At this time, anti-Mac-1, anti-Gr-1, anti-B220, anti-NK1.1, anti-CD2 and anti-TER-119 (Becton-Dickinson and PharMingen, San Diego, Calif.) antibodies were used. Positive selection was performed to separate CD117+ hematopoietic stem cells (referred as “HSC” hereinafter) from those cells obtained by negative selection by MACS using anti-CD117 antibody.

### Example 2

### Differentiation of NK Cells from HSC

The CD117+ cells obtained from bone marrow in Example were inoculated in a 24 well plate (1×10⁶ cells/well) (Falcon, USA) containing a medium supplemented with cytokine mouse SCF (30 ng/ml, PeproTech, Rocky Hill, N.J.), mouse Flt3L (50 ng/ml, PeproTech, Rocky Hill, N.J.), mouse IL-7 (5 ng/ml, PeproTech) and antibiotics [indometacin (2 ug/ml, Sigma), gentamycin (20 ug/ml, Sigma)] in a 37° C., 5% CO₂ incubator for 7 days. The medium was replaced once three days later. After 7 days of the culture, CD122⁺ premature NK cells (referred as “pNK cells” hereinafter) were isolated by MACS using FITC labeled CD122 antibody and magnetic bead conjugated anti-FITC antibody.

For differentiation into mature NK cells, the cells were further cultured in RPMI1640 medium supplemented with cytokine mouse IL-15 (50 ng/ml, PeproTech) which is important factor for NK differentiation and antibiotics [indometacin (2 ug/ml), gentamycin (20 ug/ml)] for 6 more days. Three days later, the medium was replaced. After 13 days of the culture, NK1.1⁺ cells were isolated by MACS using FITC labeled anti-NK1.1 antibody and magnetic bead conjugated anti-FITC antibody. Mature NK cells were analyzed by FACS (Fluorescence activated cell sorter) (BD Bioscience, Mountainview, Calif.) using anti-CD122, NK1.1, DX5, and NK cell receptor antibodies (FIG. 1).

### Example 3

### Preparation of OPN Knock-Out Mouse

OPN knock-out mice generated by the method described in U.S. Pat. No. 6,414,219 were purchased from Jackson Laboratory, USA.

### Example 4

### Confirmation of NK Cells in Mouse Bone Marrow and Spleen

Spleens and bones were separated from a wild-type mouse (control) and OPN knock-out mouse. The spleen was put in a strainer and ground by using a bar to prepare single cells. The bone was ground by using a mortar to isolate single cells. The spleen cells and the bone marrow cells were treated in lysis buffer (Sigma, St. Louse, Mo.) to eliminate erythrocytes. After washing with PBS, the cells were reacted with anti-NK1.1 antibody at 4° C. for 15 minutes. Then, the cells were washed and analyzed by FACS to confirm NK cells.

### Example 5

### Transplantation of Hematopoietic Cells

Donor cells were prepared by separating HSC from the control mouse and OPN knock-out mouse to compare each other. As a recipient, CD45.1 congenic mouse (Jackson Laboratory, USA) was used to distinguish it from donor cells. The recipient mouse was irradiated with gamma ray at 800 rad by using Model 109 irradiator (JL Sephered & Associates, San Fernando, Calif.). On the next day, 1×10⁶ of the donor cells (HSC) were injected through tail vein. 6 weeks later, NK cells were confirmed in the mouse spleen and lung by FACS. To compare with the recipient mouse, the control mouse and OPN knock-out mouse were irradiated by gamma ray. HSC cells obtained from CD45.2 congenic mouse were used as donor cells. To compare with the donor cells, CD45.1 congenic mouse was used as a recipient and HSC cells obtained from the control and OPN knock-out mouse were used as donor cells.

### Experimental Example 1

### Effect of Recombinant OPN on NK Differentiation

To investigate the effect of OPN on NK differentiation, in vitro differentiation of NK from HSC was induced. On the third day, recombinant OPN (2 ug/ml, R&D Systems inc., Minneapolis, Minn.) was treated to 1×10⁶ HSC cells. Then, pNK specific CD122 expression and NK specific NK1.1 expression were analyzed by FACS, followed by comparison with those of the control. As a result, it was confirmed that NK differentiation was reinforced in those cells treated with recombinant OPN, compared with the control (FIG. 2).

To confirm whether the above result was specific to recombinant OPN, the recombinant OPN was inhibited by OPN antibody (ABcam, Cambridge, UK) (FIG. 3). While inducing differentiation of NK from HSC, the antibody was treated thereto on day 3 at the concentration of 1 ug/ml one hour before the treatment of recombinant OPN. Three days later, inhibition of recombinant OPN was confirmed. Expression changes of CD122 and NK1.1, the molecules related to NK, by recombinant OPN were investigated by RT-PCR at RNA level. First, RNA was extracted from HSC, pNK and mNK using Trizol reagent (Invitrogen, Carlsbad, Calif.). 3 ug of the RNA was incubated with Moloney murine leukemia virus reverse transcriptase (Roche) at 37° C. for 1 hour to synthesize cDNA. The synthesized cDNA proceeded to PCR using Maxime PCR Premix kit (Intron, Korea) as follows: at 95° C. for 1 minute, at 55° C. for 1 minute, at 72° C. for 2 minutes (28 cycles or 32 cycles) and then at 72° C. for 10 minutes. The amplified PCR product proceeded to electrophoresis, followed by staining with EtBr. Real-Time PCR was also performed using SYBR Premix Ex Tag (TaKaRa, Tokyo, Japan) with Dice™ TP 800 Thermal Cycler (TaKaRa). According to the manufacturer's instruction, CD122 RNA and NK1.1 RNA were separated. cDNA was synthesized by real-time PCR using real-time PCR kit (Quiagen, Germany) according to the manufacturer's instruction. PCR mixture containing the synthesized cDNA was heated at 95° C. for 1 minute, followed by PCR. PCR with HSC or mNK was also performed as follows: at 95° C. for 1 minute, at 55° C. for 1 minute, and at 72° C. for 2 minutes (28 or 32 cycles). PCR with pNK was performed as follows: at 95° C. for 1 minute, at 60° C. for 1 minute, at 72° C. for 2 minutes (28 or 32 cycles) and then at 72° C. for 10 minutes for final extension. The amplified PCR products proceeded to electrophoresis, followed by staining with EtBr.

As a result, expressions of CD122 and NK1.1 were increased by recombinant OPN (FIG. 4). Therefore, it was confirmed that OPN was involved in NK differentiation.

### Experimental Example 2

### Effect of Recombinant OPN on Cytotoxic Activity of NK

Differentiated NK cells were treated with IL-2 (10 u/ml), followed by culture for 24 hours. The cultured NK cells were washed and distributed in a 96 well round bottom plate (Falcon, USA) together with ⁵¹Cr-labeled YAC-1 cells (10⁴ cells/well) according to the ratio of effector cells to target cells, followed by culture in a 37° C., 5% CO₂ incubator for 4 hours. Supernatant was obtained, followed by ⁵¹Cr release assay using γ-counter.

As a result, cytotoxic effect of NK was increased by recombinant OPN (FIG. 5). Therefore, OPN was confirmed to be involved in NK activity.

### Experimental Example 3

### Differentiation of NK Cells in OPN Knock-Out Mouse

NK1.1+CD3-NK cells were confirmed by FACS in the control and OPN knock-out mouse. As a result, it was confirmed that NK cells were decreased in OPN knock-out mouse spleen and bone marrow (FIG. 6).

The expressions of LY49s which is largely expressed in mature NK cells in the control mouse spleen cells and bone marrow cells using LY49 antibodies by FACS. As a result, LY49 receptor expression was significantly reduced in OPN knock-out mouse (FIG. 7).

The expressions of NK cell specific molecules were also investigated by RT-PCR at RNA level. As a result, the expressions of NK specific perforin and granzyme were reduced (FIG. 8). Therefore, it was confirmed that OPN deficiency resulted in the inhibition of NK cells.

### Experimental Example 4

### Effect of Paracrine OPN on NK Cell Differentiation

The cells of each differentiation stage obtained by in vivo differentiation were separated by using FACSAria, followed by real-time PCR to analyze them at RNA level. As a result, OPN expressions in the cells were not much different (FIG. 9). The cells of each differentiation stage obtained by in vitro differentiation were separated, followed by ELISA to confirm OPN expression. As a result, OPN level was higher in mNK and particularly OPN expression in stromal cells was significantly increased (FIG. 10). Thus, it was confirmed that intracellular OPN expression did not affect cell differentiation but OPN secreted by the cells involved in NK cell differentiation could affect NK cell differentiation.

HSC obtained from OPN knock-out mouse was differentiated in vitro, which was compared with that of the control. As a result, the HSC was as fully differentiated as the control mouse HSC was. And NK differentiation was increased by the treatment of recombinant OPN (FIG. 11A). On the other hand, when OPN knock-out stromal cells were co-cultured with HSC, NK differentiation from the OPN knock-out stromal cells was inhibited (FIG. 11B).

HSC (donor cells) obtained from a wild-type mouse was transplanted in a wild type mouse and OPN knock-out mouse (recipient mice), followed by differentiation for 6 weeks. Then, NK differentiation was investigated. As a result, HSC transplanted in OPN knock-out mouse was significantly prevented from being differentiated to NK cells in spleen and lung (FIG. 11C).

HSC (donor cells) obtained from a wild-type mouse and OPN knock-out mouse was transplanted in a wild type mouse (recipient mouse), and then NK differentiation was induced. As a result, NK differentiations in the wild-type mouse transplanted with OPN knock-out mouse originated HSC and the control mouse originated HSC were not much different (FIG. 11D). Therefore, NK cell differentiation was believed to be affected by OPN secreted in foreign environment.

### Experimental Example 5

### OPN Effect Confirmed by T-bet

Recombinant OPN was treated to HSC obtained from the mouse bone marrow during in vitro NK differentiation, followed by RT-PCR and real-time PCR to investigate T-bet expression. As a result, T-bet expression was increased (FIG. 12) but decreased in OPN knock-out mouse (FIG. 13). To investigate CD122 expression by T-bet, 293T cells were transformed with CD122 promoter conjugated luciferase plasmid and T-bet conjugated plasmid, followed by cell lysis. The cell lysate was reacted with luciferase the matrix of luciferase, followed by analysis. As a result, T-bet regulated CD122 (FIG. 14) and precisely T-bet increased by OPN could regulate CD122 expression to reinforce NK cell differentiation.

To confirm the above result, HSC separated from T-bet knock-out mouse (Jackson Laboratory, U.S.A) was differentiated and NK differentiation was compared with that of the control. The differentiated cells were treated with recombinant OPN to investigate how T-bet was involved in NK differentiation induced by OPN by FACS. As a result, NK differentiation was not affected by OPN (FIG. 15).

The cells under the differentiation were directly obtained from the T-bet knock-mouse bone marrow and CD122 expression therein was compared with that of the control. The cells were treated with recombinant OPN to induce NK differentiation. Then, T-bet involvement in OPN mediated NK differentiation was investigated by FACS. As a result, OPN effect was not observed in those cells not differentiated, directly obtained from a wild-type mouse, unlike in the control (FIG. 16). Therefore, OPN was confirmed to affect NK cell differentiation via T-bet.

NK cells were separated from the wild-type mouse spleen, T-bet knock-out mouse spleen and OPN knock-out mouse spleen. Immature NK cells were tested for CD122 expression by FACS. As a result, OPN did not affect mature NK cells but affected immature cells not differentiated yet (FIG. 17).

The Manufacturing Examples of the composition for the present invention are described hereinafter.

### Manufacturing Example 1

### Preparation of Pharmaceutical Formulations

### <1-1> Preparation of Powders

| | |
|---|---|
| OPN | 2 g |
| Lactose | 1 g |

Powders were prepared by mixing all the above components, which were filled in airtight packs according to the conventional method for preparing powders.

### <1-2> Preparation of Tablets

| | | |
|---|---|---|
| OPN | 100 | mg |
| Corn starch | 100 | mg |
| Lactose | 100 | mg |
| Magnesium stearate | 2 | mg |

Tablets were prepared by mixing all the above components by the conventional method for preparing tablets.

### <1-3> Preparation of Capsules

| | | |
|---|---|---|
| OPN | 100 | mg |
| Corn starch | 100 | mg |
| Lactose | 100 | mg |
| Magnesium stearate | 2 | mg |

Capsules were prepared by mixing all the above components, which were filled in gelatin capsules according to the conventional method for preparing capsules.

### <1-4> Preparation of Pills

| | | |
|---|---|---|
| OPN | 1 | g |
| Lactose | 1.5 | g |
| Glycerin | 1 | g |
| Xylitol | 0.5 | g |

Pills were prepared by mixing all the above components according to the conventional method for preparing pills. Each pill contained 4 g of the mixture.

### <1-5> Preparation of Granules

| | | |
|---|---|---|
| OPN | 150 | mg |
| Soybean extract | 50 | mg |
| Glucose | 200 | mg |
| Starch | 600 | mg |

All the above components were mixed, to which 100 mg of 30% ethanol was added. The mixture was dried at 60° C. and the prepared granules were filled in packs.

### <1-6> Preparation of Injectable Solutions

| | | |
|---|---|---|
| OPN | 10 | μg/ml |
| Weak HCl BP | until pH | 7.6 |
| Injectable NaCl BP | up to 1 | ml |

OPN was dissolved in proper volume of injectable NaCl BP. pH of the prepared solution was regulated as 7.6 by using weak HCl BP. The volume was adjusted by using injectable NaCl BP. The solution was well mixed and filled in 5 ml type I transparent glass ampoules. The ampoules were sealed by melting the glass of opening, followed by autoclave at 120° C. for at least 15 minutes for sterilization.

### Manufacturing Example 2

### Preparation of Food

The powders, tablets, capsules, pills and granules prepared in Manufacturing Example 1 can be applied to food. Foods containing OPN were prepared as follows.

### <2-1> Preparation of Flour Food

0.1˜10.0 weight part of OPN was added to the flour. Health enhancing foods such as bread, cake, cookies, crackers and noodles were prepared with the flour mixture according to the conventional method.

### <2-2> Preparation of Soups and Gravies

0.1˜1.0 weight part of OPN was added to soups and gravies. Health enhancing meat products, soups and gravies were prepared with this mixture by the conventional method.

### <2-3> Preparation of Ground Beef

Health enhancing ground beef was prepared by mixing 10 weight part of OPN with ground beef according to the conventional method.

### <2-4> Preparation of Dairy Products

0.1˜1.0 weight part of OPN was added to milk. Health enhancing dairy products such as butter and ice cream were prepared with the milk mixture according to the conventional method.

### <2-5> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

OPN was concentrated under reduced pressure, spray-dried and pulverized to obtain 60-mesh dry powders.

Sun-Sik was prepared by mixing the dry powders of the grains, seeds and OPN according to the below ratio.

Grains (brown rice: 30 weight part, Yulmu: 15 weight part, barley: 20 weight part),

Seeds (wild sesame: 7 weight part, black soybean: 8 weight part, black sesame: 7 weight part),

OPN (1 weight part),

Ganoderma lucidum (0.5 weight part),

Rehmannia glutinosa (0.5 weight part)

### Manufacturing Example 3

### Preparation of Beverages

Beverages containing OPN were prepared as follows.

### <3-1> Preparation of Health Beverages

OPN (0.5 weight part) was mixed with liquid fructose (0.5 weight part), oligosaccharide (2 weight part), sugar (2 weight part), salt (0.5 weight part), and water (75 weight part). After mixing completely, the mixture was sterilized instantly and filled small containers such as glass bottles, pet bottles, etc, to prepare health beverages.

### <3-2> Preparation of Vegetable Juice

Health enhancing vegetable juice was prepared by adding 0.5 g of OPN to 1,000 ml of tomato or carrot juice according to the conventional method.

### <3-3> Preparation of Fruit Juice

Health enhancing fruit juice was prepared by adding 0.1 g of OPN to 1,000 ml of apple or grape juice according to the conventional method.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1-5. (canceled)

6. A method for accelerating differentiation of natural killer cells from hematopoietic stem cells comprising the following steps:
1) inducing proliferation of premature natural killer cells adding a premature natural killer cell inducer to hematopoietic stem cells; and
2) inducing differentiation to mature natural killer cells by adding osteopontin to the premature natural killer cells of step 1).

7. The method according to claim 6, wherein the premature natural killer cell inducer of step 1) is SCF, Flt3L or IL-7.

8. The method according to claim 6, wherein the premature natural killer cells of step 2) are co-cultured with IL-15 (Interleukin-15).

9. A method for differentiation of natural killer cells having improved cytotoxic activity comprising the following steps:
1) inducing proliferation of premature natural killer cells adding premature natural killer cell inducer to hematopoietic stem cells; and
2) inducing differentiation to mature natural killer cells by adding osteopontin to the premature natural killer cells of step 1).

10. The method according to claim 9, wherein the premature natural killer cell inducer of step 1) is SCF, Flt3L or IL-7.

11. The method according to claim 9, wherein the premature natural killer cells of step 2) are co-cultured with IL-15 (Interleukin-15).

12. A method for treating cancer containing the step of administering the natural killer cells having improved cytotoxic activity by the method of claim 9 to a subject with cancer.

13. A method for preventing cancer containing the step of administering the natural killer cells having improved cytotoxic activity by the method of claim 9 to a subject.

14. The method for preventing or treating cancer according to claim 12, wherein the cancer is selected from the group consisting of breast cancer, melanoma, stomach cancer, liver cancer and lung cancer.

15-19. (canceled)

20. The method for preventing or treating cancer according to claim 13, wherein the cancer is selected from the group consisting of breast cancer, melanoma, stomach cancer, liver cancer and lung cancer.
